(19) United States
(12) Patent Application Publication
(10) Pub. No.: US 2011/0152150 A1
(43) Pub. Date: Jun. 23, 2011

(54) CLEANSING COMPOSITION

(75) Inventor: Anne-Laure BERNARD, Antony (FR)

(73) Assignee: L'OREAL, Paris (FR)

(21) Appl. No.: 12/969,842

(22) Filed: Dec. 16, 2010

Related U.S. Application Data
(60) Provisional application No. 61/298,218, filed on Jan. 26, 2010.

(30) Foreign Application Priority Data
Dec. 22, 2009 (FR) 09 59371

(51) Int. Cl. A61Q 1/14; A61Q 19/10; A61K 8/37; A61K 8/36; A61K 8/64; A61K 8/55; A61K 8/46; A61K 8/44; A61K 8/60; A61K 8/92; A61K 8/73

(52) U.S. Cl. 510/136; 510/130; 132/200

(57) ABSTRACT

Composition containing at least one foaming anionic or amphoteric surfactant and at least one compound derived from jasmonic acid. Methods of use.

### REFERENCE TO PRIOR APPLICATIONS

This application claims priority to U.S. provisional application Ser. No. 61/298,218, filed Jan. 26, 2010; and to French patent application 09 59371, filed Dec. 22, 2009, both incorporated herein by reference.

### FIELD OF THE INVENTION

The invention relates to a cleansing composition comprising, in an aqueous medium, a jasmonic acid derivative and a foaming anionic or amphoteric surfactant, and to its uses in cleansing and/or removing makeup from keratinous substances, such as the skin, mucous membranes, lips and/or keratinous fibres, such as the eyelashes or hair.

### BACKGROUND OF THE INVENTION

Cleansing the skin is very important in caring for the face. It must be as efficient as possible because greasy residues, such as excess sebum, the remnants of cosmetic products used daily and makeup products, accumulate in the folds of the skin and can block the pores of the skin and result in the appearance of spots. One means for properly cleansing the skin is to use foaming cleansing products.

One of the major difficulties in the formulation of foaming products consists in developing products having a very good foaming power, the foaming performances generally being related by the consumers to efficient cleansing. For the formulation of a product of such a quality, it is necessary to use foaming surfactants in order to provide a fast, copious and dense foam (which does not run over the face) which is easy to spread and which will make possible good rinsing.

### DETAILED DESCRIPTION OF THE PREFERRED EMBODIMENTS

The inventor has discovered, surprisingly, that the use of a jasmonic acid derivative in combination with a foaming anionic and/or amphoteric surfactant makes it possible to improve the qualities of the foam of cleansing compositions.

Thus, a subject-matter of the invention is a composition comprising (1) at least one foaming anionic and/or amphoteric surfactant and (2) at least one compound derived from jasmonic acid chosen from those corresponding to the following formula (I):

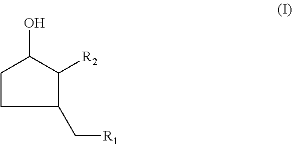

in which:

R₁ represents a COOR₃ radical, R₃ denoting a hydrogen atom or a C₁-C₄ alkyl radical optionally substituted by one or more hydroxyl groups;

R₂ represents a hydrocarbon radical which is saturated or unsaturated, which is linear and which has from 1 to 18 carbon atoms or which is branched or cyclic and which has from 3 to 18 carbon atoms;

and their optical isomers, and corresponding salts.

The composition of the invention exhibits a good foaming power, in particular a dense and copious foam, while being thoroughly rinsable.

As the composition of the invention is preferably a cosmetic composition, it preferably comprises a physiologically acceptable medium. The term “physiologically acceptable medium” is understood here to mean a medium compatible with keratinous substances. It is also preferably a cosmetically acceptable medium, that is to say which additionally exhibits a pleasant colour, a pleasant odour and pleasant feel and which does not generate unacceptable discomfort (smarting, tightness, redness) liable to dissuade the consumer from using this composition.

The cleansing composition of the invention is a foaming composition which is rinsed off after application to the skin.

### Jasmonic Acid Derivative

The compound which is a jasmonic acid derivative is a compound chosen from those corresponding to the following formula (I):

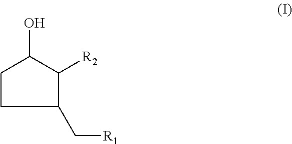

in which:
R₁ represents a COOR₃ radical, R₃ denoting a hydrogen atom or a C₁-C₄ alkyl radical optionally substituted by one or more hydroxyl groups;
R₂ represents a hydrocarbon radical which is saturated or unsaturated, which is linear and which has from 1 to carbon atoms or which is branched or cyclic and which has from 3 to 18 carbon atoms;
and their optical isomers, and corresponding salts.

Preferably, R₁ denotes a radical chosen from —COOH, —COOMe, —COO—CH₂—CH₃, —COO—CH₂—CH(OH)—CH₂OH, —COOCH₂—CH₂—CH₂OH or —COOCH₂—CH(OH)—CH₃. Preferably R₁ denotes a —COOH radical.

Preferably, R₂ denotes a saturated or unsaturated linear hydrocarbon radical preferably having from 2 to carbon atoms. In particular, R₂ can be a pentyl, pentenyl, hexyl or heptyl radical.

According to one embodiment, the compound of formula (I) is chosen from 3-hydroxy-2-[(2Z)-2-pentenyl]cyclopentaneacetic acid or 3-hydroxy-2-pentylcyclopentaneacetic acid and is preferably 3-hydroxy-2-pentylcyclopentaneacetic acid.

The salts of the compounds which can be used according to the invention are chosen in particular from alkali metal salts, for example sodium or potassium salts; alkaline earth metal salts, for example calcium, magnesium or strontium salts; metal salts, for example zinc, aluminium, manganese or copper salts; salts of ammonium of formula NH₄⁺; quaternary ammonium salts; organic amine salts, such as, for example, methylamine, dimethylamine, trimethylamine, triethylamine, ethylamine, 2-hydroxyethylamine, bis(2-hydroxyethyl)amine or tris(2-hydroxyethyl)amine salts; or lysine or arginine salts. Use is preferably made of salts chosen from sodium, potassium, calcium, magnesium, strontium, copper, manganese or zinc salts.

The compound derived from jasmonic acid of formula (I) can be present in an amount ranging for example from 0.1 to 15% by weight, preferably from 0.2 to 10% by weight and better still from 0.5 to 5% by weight, with respect to the total weight of the composition.

### Anionic and Amphoteric Surfactants

The anionic and amphoteric (or zwitterionic) surfactants present in the composition according to the invention are preferably foaming surfactants capable of cleansing the skin.

Foaming surfactants are detergents and differ from emulsifiers in the value of their HLB (Hydrophilic-Lipophilic Balance), the HLB being the ratio of the hydrophilic part to the lipophilic part in the molecule. The term “HLB” is well known to the person skilled in the art and is described, for example, in “The HLB system. A time-saving guide to Emulsifier Selection” (published by ICI Americas Inc, 1984). For emulsifiers, HLB generally ranges from 3 to 8 for the preparation of W/O emulsions and from 8 to 18 for the preparation of O/W emulsions, whereas foaming surfactants generally have an HLB of greater than 20.

The foaming anionic and/or amphoteric surfactants can be present in the composition in an amount (as active material) ranging, for example, from 0.1 to 40% by weight, preferably from 0.5 to 30% by weight, better still from 1 to 25% by weight and even better still from 3 to 20% by weight, with respect to the total weight of the composition.

### Anionic Surfactants

The anionic surfactants which can be added to the composition according to the invention can be chosen in particular from anionic derivatives of proteins of vegetable origin or of silk proteins, phosphates and alkyl phosphates, carboxylates, sulphosuccinates, amino acid derivatives, alkyl sulphates, alkyl ether sulphates, sulphonates, isethionates, taurates, alkyl sulphoacetates, polypeptides, anionic derivatives of alkyl polyglucosides, soaps (salts of fatty acids), and their mixtures.

a) Anionic derivatives of proteins of vegetable origin are protein hydrolysates comprising a hydrophobic group, it being possible for the hydrophobic group to be naturally present in the protein or to be added by reaction of the protein and/or of the protein hydrolysate with a hydrophobic compound. The proteins are of vegetable origin or derived from silk, and the hydrophobic group can in particular be a fatty chain, for example an alkyl chain comprising from 10 to 22 carbon atoms. Mention may more particularly be made, as anionic derivatives of proteins of vegetable origin, of apple, wheat, soybean or oat protein hydrolysates comprising an alkyl chain having from 10 to 22 carbon atoms, and their salts. The alkyl chain can in particular be a lauryl chain and the salt can be a sodium, potassium and/or ammonium salt.

Thus, mention may be made, as protein hydrolysates comprising a hydrophobic group, for example, of salts of protein hydrolysates where the protein is a silk protein modified by lauric acid, such as the product sold under the name Kawa Silk by Kawaken; salts of protein hydrolysates where the protein is a wheat protein modified by lauric acid, such as the potassium salt sold under the name Aminofoam W OR by Croda (CTFA name: potassium lauroyl wheat amino acids) and the sodium salt sold under the name Proteol LW 30 by Seppic (CTFA name: sodium lauroyl wheat amino acids); salts of protein hydrolysates where the protein is an oat protein comprising an alkyl chain having from 10 to 22 carbon atoms and more especially salts of protein hydrolysates where the protein is an oat protein modified by lauric acid, such as the sodium salt sold under the name Proteol OAT (30% aqueous solution) by Seppic (CTFA name: sodium lauroyl oat amino acids); or salts of apple protein hydrolysates comprising an alkyl chain having from 10 to 22 carbon atoms, such as the sodium salt sold under the name Proteol APL (30% aqueous/glycol solution) by Seppic (CTFA name: sodium cocoyl apple amino acids). Mention may also be made of the mixture of lauroyl amino acids (aspartic acid, glutamic acid, glycine, alanine) neutralized with sodium N-methylglycinate sold under the name Proteol SAV 50 S by Seppic (CTFA name: sodium cocoyl amino acids).

b) Mention may be made, as phosphates and alkyl phosphates, for example, of monoalkyl phosphates and dialkyl phosphates, such as lauryl monophosphate, sold under the name MAP 20® by Kao Chemicals, the potassium salt of dodecyl phosphate, the mixture of mono- and diesters (predominantly diester) sold under the name Crafol AP-31® by Cognis, the mixture of octyl phosphate monoester and diester, sold under the name Crafol AP-20® by Cognis, the mixture of ethoxylated (7 mol of EO) 2-butyloctyl phosphate monoester and diester, sold under the name Isofol 12 7 EO-Phosphate Ester® by Condea, the potassium or triethanolamine salt of mono(C₁₂-C₁₃)alkyl phosphate, sold under the references Arlatone MAP 230K-40® and Arlatone MAP 230T-60® by Uniqema, potassium lauryl phosphate, sold under the name Dermalcare MAP XC-99/09® by Rhodia Chimie, and potassium cetyl phosphate, sold under the name Arlatone MAP 160K by Uniqema.

c) Mention may be made, as carboxylates, of:
amido ether carboxylates (AEC), such as sodium lauryl amido ether carboxylate (3 EO), sold under the name Akypo Foam 30® by Kao Chemicals.
polyoxyethylenated carboxylic acid salts, such as oxyethylenated (6 EO) sodium lauryl ether carboxylate (65/25/10 C₁₂-C₁₄-C₁₆), sold under the name Akypo Soft 45 NV® by Kao Chemicals, polyoxyethylenated and carboxymethylated fatty acids originating from olive oil, sold under the name Olivem 400® by Biologia E Tecnologia, or oxyethylenated (6 EO) sodium tridecyl ether carboxylate, sold under the name Nikkol ECTD-6NEX® by Nikkol.
salts of fatty acids (soaps) having a C₆ to C₂₂ alkyl chain which are neutralized with an organic or inorganic base, such as potassium hydroxide, sodium hydroxide, triethanolamine, N-methylglucamine, lysine and arginine.

d) Mention may in particular be made, as amino acid derivatives, of alkali salts of amino acids, such as:
sarcosinates, such as sodium lauroyl sarcosinate, sold under the name Sarkosyl NL 97® by Ciba or sold under the name Oramix L 30® by Seppic, sodium myristoyl sarcosinate, sold under the name Nikkol Sarcosinate MN® by Nikkol, or sodium palmitoyl sarcosinate, sold under the name Nikkol Sarcosinate PN® by Nikkol.
alaninates, such as sodium N-lauroyl-N-methylamidopropionate, sold under the name Sodium Nikkol Alaninate LN 30® by Nikkol or sold under the name Alanone ALE® by Kawaken, or triethanolamine N-lauroyl-N-methylalanine, sold under the name Alanone ALTA® by Kawaken.
glutamates, such as triethanolamine monococoyl glutamate, sold under the name Acylglutamate CT-12® by Ajinomoto, triethanolamine lauroyl glutamate, sold under the name Acylglutamate LT-12® by Ajinomoto.
aspartates, such as the mixture of triethanolamine N-lauroyl aspartate and triethanolamine N-myristoyl aspartate, sold under the name Asparack® by Mitsubishi.
glycine derivatives (glycinates), such as sodium N-cocoyl glycinate, sold under the names Amilite GCS-12® and Amilite GCK 12 by Ajinomoto.
citrates, such as the citric monoester of oxyethylenated (9 mol) coco alcohols, sold under the name Witconol EC 1129 by Goldschmidt.
galacturonates, such as sodium dodecyl D-galactoside uronate, sold by Soliance.

e) Mention may be made, as sulphosuccinates, for example, of oxyethylenated (3 EO) lauryl (70/30 C₁₂/C₁₄) alcohol monosulphosuccinate, sold under the names Setacin 103 Special® and Rewopol SB-FA 30 K 4® by Witco, the disodium salt of a hemisulphosuccinate of C₁₂-C₁₄ alcohols, sold under the name Setacin F Special Paste® by Zschimmer Schwarz, oxyethylenated (2 EO) disodium oleamidosulphosuccinate, sold under the name Standapol SH 135® by Cognis, oxyethylenated (5 EO) lauramide monosulphosuccinate, sold under the name Lebon A-5000® by Sanyo, the disodium salt of oxyethylenated (10 EO) lauryl citrate monosulphosuccinate, sold under the name Rewopol SB CS 50® by Witco, or ricinoleic monoethanolamide monosulphosuccinate, sold under the name Rewoderm S 1333® by Witco. Use may also be made of polydimethylsiloxane sulphosuccinates, such as disodium PEG-12 dimethicone sulphosuccinate, sold under the name Mackanate-DC 30 by MacIntyre.

f) Mention may be made, as alkyl sulphates, for example, of triethanolamine lauryl sulphate (CTFA name: TEA lauryl sulphate), such as the product sold by Huntsman under the name Empicol TL40 FL or the product sold by Cognis under the name Texapon T42, which products are at 40% in aqueous solution. Mention may also be made of ammonium lauryl sulphate (CTFA name: ammonium lauryl sulphate), such as the product sold by Huntsman under the name Empicol AL 30FL, which is at 30% in aqueous solution.

g) Mention may be made, as alkyl ether sulphates, for example, of sodium lauryl ether sulphate (CTFA name: sodium laureth sulphate), such as that sold under the names Texapon N40 and Texapon AOS 225 UP by Cognis, or ammonium lauryl ether sulphate (CTFA name: ammonium laureth sulphate), such as that sold under the name Standapol EA-2 by Cognis.

h) Mention may be made, as sulphonates, for example, of α-olefinsulphonates, such as sodium α-olefinsulphonate (C₁₄-C₁₆), sold under the name Bio-Terge AS-40® by Stepan, sold under the names Witconate AOS Protégé® and Sulframine AOS PH 12® by Witco or sold under the name Bio-Terge AS-40 CG® by Stepan, secondary sodium olefinsulphonate, sold under the name Hostapur SAS 30® by Clariant; or linear alkylarylsulphonates, such as sodium xylenesulphonate, sold under the names Manrosol SXS30®, Manrosol SXS40® and Manrosol SXS93® by Manro.

i) Mention may be made, as isethionates, of acylisethionates, such as sodium cocoylisethionate, such as the product sold under the name Jordapon CI P® by Jordan.

j) Mention may be made, as taurates, of the sodium salt of palm kernel oil methyltaurate, sold under the name Hostapon CT Paté® by Clariant; N-acyl-N-methyltaurates, such as sodium N-cocoyl-N-methyltaurate, sold under the name Hostapon LT-SF® by Clariant or sold under the name Nikkol CMT-30-T® by Nikkol, or sodium palmitoyl methyltaurate, sold under the name Nikkol PMT® by Nikkol.

k) The anionic derivatives of alkyl polyglucosides can in particular be citrates, tartrates, sulphosuccinates, carbonates and glycerol ethers obtained from alkyl polyglucosides. Mention may be made, for example, of the sodium salt of cocoylpolyglucoside (1,4) tartaric ester, sold under the name Eucarol AGE-ET® by Cesalpinia, the disodium salt of cocoylpolyglucoside (1,4) sulphosuccinic ester, sold under the name Essai 512 MP® by Seppic, or the sodium salt of cocoylpolyglucoside (1,4) citric ester, sold under the name Eucarol AGE-EC® by Cesalpinia.

1) The soaps are obtained from a fatty acid which is partially or completely saponified (neutralized) by a basic agent. These are soaps of alkali metals or alkaline earth metals or of organic bases. Use may be made, as fatty acids, of saturated linear or branched fatty acids comprising from 8 to 30 carbon atoms and preferably comprising from 8 to 22 carbon atoms. This fatty acid can be chosen in particular from palmitic acid, stearic acid, myristic acid, lauric acid and their mixtures.

Use may be made, as basic agents, for example, of alkali metal hydroxides (sodium hydroxide and potassium hydroxide), alkaline earth metal hydroxides (for example magnesium hydroxide), ammonium hydroxide or organic bases, such as triethanolamine, N-methylglucamine, lysine and arginine.

The soaps can in particular be fatty acid alkali metal salts, the basic agent being an alkali metal hydroxide, preferably potassium hydroxide (KOH).

The amount of basic agent must be sufficient for the fatty acid to be at least partially neutralized.

Preferably, the anionic surfactant is chosen from alkyl sulphates, alkyl ether sulphates, such as sodium lauryl ether sulphate, isethionates, amino acid derivatives, in particular glycine derivatives (glycinates), such as sodium N-cocoylglycinate, and their mixtures.

### Foaming Amphoteric and Zwitterionic Surfactants

The amphoteric and zwitterionic surfactants can be chosen, for example, from betaines, N-alkyl-amidobetaines and their derivatives, sultaines, alkyl polyaminocarboxylates, alkylamphoacetates, and their mixtures. As used herein, the term amphoteric surfactants includes zwitterionic surfactants unless particularly specified to the contrary.

Mention may in particular be made, as betaines, of alkyl betaines, such as, for example, coco betaine, such as the product sold under the name Dehyton AB-30® by Cognis, lauryl betaine, such as the product sold under the name Genagen KB® by Clariant, oxyethylenated (10 EO) lauryl betaine, such as the product sold under the name Lauryl Ether (10 OE) Betaine® by Shin Nihon Rica, or oxyethylenated (10 EO) stearyl betaine, such as the product sold under the name Stearyl Ether (10 OE) Betaine® by Shin Nihon Rica.

Mention may be made, among N-alkylamidobetaines and their derivatives, for example, of cocamidopropyl betaine, sold under the name Lebon 2000 HG® by Sanyo or sold under the name Empigen BB® by Albright & Wilson, or lauramidopropyl betaine, sold under the name Rewoteric AMB12® by Witco.

Mention may be made, as sultaines, of hydroxysultaines, such as cocamidopropyl hydroxysultaine, such as the product sold under the name Rewoteric AM CAS by Goldschmidt-Degussa or the product sold under the name Crosultaine C-50® by Croda.

Mention may be made, as alkyl polyaminocarb-oxylates (APACs), of sodium cocoylpolyaminocarboxylate, sold under the names Ampholak 7 CX/C® and Ampholak 7 CX® by Akzo Nobel, sodium stearylpolyamidocarboxylate, sold under the name Ampholak 7 TX/C by Akzo Nobel, or sodium carboxymethyloleylpolypropylamine, sold under the name Ampholak XO7/C® by Akzo Nobel.

Mention may be made, as alkylamphoacetates, for example, of N-disodium N-cocoyl-N-carboxymethoxyethyl-N-(carboxymethyl)ethylenediamine (CTFA name: disodium cocoamphodiacetate), such as the product sold under the name Miranol C2M Concentré NP® by Rhodia, N-sodium N-cocoyl-N-hydroxyethyl-N-(carboxymethyl)ethylene-diamine (CTFA name: sodium cocamphoacetate) or sodium cocoamphohydroxypropylsulphonate, sold under the name Miranol CSE by Rhodia.

Preferably, the amphoteric or zwitterionic surfactant is chosen from betaines, in particular alkyl betaines, alkylamphoacetates, such as sodium cocamphoacetate, and their mixtures.

The composition according to the invention can comprise, in addition to the foaming anionic and/or amphoteric surfactants, one or more additional foaming surfactants chosen from nonionic surfactants, cationic surfactants and their mixtures.

### Foaming Nonionic Surfactant

The foaming nonionic surfactants of the composition of the invention can be chosen in particular from alkyl polyglucosides (APGs), oxyalkylenated glycerol esters, oxyalkylenated sugar esters and their mixtures. These are preferably APGs.

Use is preferably made, as alkyl polyglucosides, of those comprising an alkyl group comprising from 6 to 30 carbon atoms and preferably from 8 to 16 carbon atoms and comprising a glucoside group preferably comprising 1.2 to 3 glucoside units. The alkyl polyglucosides can be chosen, for example, from decyl glucoside ((C₉/C₁₁ alkyl) polyglucoside (1.4)), such as the product sold under the name Mydol 10® by Kao Chemicals, or the product sold under the name Plantacare 2000 UP® by Cognis; caprylyl/capryl glucoside, such as the product sold under the name Plantacare KE 3711® by Cognis; lauryl glucoside, such as the product sold under the name Plantacare 1200 UP® by Cognis; coco glucoside, such as the product sold under the name Plantacare 818 UP® by Cognis; caprylyl glucoside, such as the product sold under the name Plantacare 810 UP® by Cognis; and their mixtures.

The oxyalkylenated glycerol esters are in particular the polyoxyethylenated derivatives of esters of glycerol and of fatty acids and of their hydrogenated derivatives. These oxyalkylenated glycerol esters can be chosen, for example, from esters of glycerol and of fatty acids which are hydrogenated and oxyethylenated, such as PEG-200 hydrogenated glyceryl palmate, sold under the name Rewoderm LI-S 80 by Goldschmidt; oxyethylenated glyceryl cocoates, such as PEG-7 glyceryl cocoate, sold under the name Tegosoft GC by Goldschmidt, and PEG-30 glyceryl cocoate, sold under the name Rewoderm LI-63 by Goldschmidt; and their mixtures.

The oxyalkylenated sugar esters are in particular polyethylene glycol ethers of fatty acid and sugar esters. These oxyalkylenated sugar esters can be chosen, for example, from oxyethylenated glucose esters, such as PEG-120 methyl glucose dioleate, sold under the name Glucamate DOE 120 by Amerchol.

According to a preferred embodiment of the invention, the nonionic surfactant is an alkyl polyglucoside which can be chosen in particular from decyl glucoside, caprylyl/capryl glucoside, lauryl glucoside, coco glucoside, caprylyl glucoside and their mixtures.

### Foaming Cationic Surfactant

According to one embodiment, the composition according to the invention can comprise at least one cationic surfactant, in particular in the case where it comprises a foaming amphoteric surfactant. The cationic surfactants which can be used according to the present invention are in particular salts of optionally polyoxyalkylenated primary, secondary or tertiary fatty amines; quaternary ammonium salts; imidazoline derivatives; amine oxides having a cationic nature; and/or one of their mixtures.

The quaternary ammonium salts are, for example:
those which exhibit the following general formula (IV):

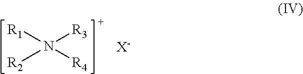

in which the R₁ to R₄ radicals, which can be identical or different, represent a linear or branched aliphatic radical comprising from 1 to 30 carbon atoms or an aromatic radical, such as an aryl or alkylaryl. The aliphatic radicals can comprise heteroatoms, such as, in particular, oxygen, nitrogen or sulphur, or halogens. The aliphatic radicals are chosen, for example, from alkyl, alkoxy, polyoxy(C₂-C₆)alkylene, alkylamide, (C₁₂-C₂₂)alkylamido(C₂-C₆)alkyl, (C₁₂-C₂₂)alkyl acetate or hydroxyalkyl radicals comprising approximately from 1 to 30 carbon atoms; X is an anion chosen from the group of the halides, phosphates, acetates, lactates, (C₂-C₆)alkyl sulphates, or alkyl- or alkylarylsulphonates. Preferably, R₁ and R₂ denote a C₁-C₄ alkyl or a C₁-C₄ hydroxyalkyl.
imidazolinium quaternary ammonium salts, such as, for example, that of following formula (V):

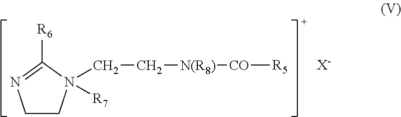

in which R₅ represents an alkenyl or alkyl radical comprising from 8 to 30 carbon atoms, for example derived from coconut fatty acids, R₆ represents a hydrogen atom, a C₁-C₄ alkyl radical or an alkenyl or alkyl radical comprising from 8 to 30 carbon atoms, R₇ represents a C₁-C₄ alkyl radical, R₈ represents a hydrogen atom or a C₁-C₄ alkyl radical and X⁻ is an anion chosen from the group of the halides, phosphates, acetates, lactates, alkyl sulphates, or alkyl- or alkylarylsulphonates. Preferably, R₅ and R₆ denote a mixture of alkenyl or alkyl radicals comprising from 12 to 21 carbon atoms, for example derived from tallow fatty acids, R₇ denotes methyl and R₈ denotes hydrogen.
di(quaternary ammonium) salts of formula (VI):

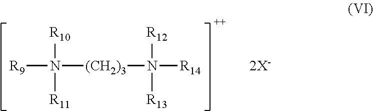

in which R₉ denotes an aliphatic radical comprising approximately from 16 to 30 carbon atoms, R₁₀, R₁₁, R₁₂, R₁₃ and R₁₄, which are identical or different, are chosen from hydrogen or an alkyl radical comprising from 1 to 4 carbon atoms and X is an anion chosen from the group of the halides, acetates, phosphates, nitrates and methyl sulphates.
quaternary ammonium salts comprising at least one ester functional group, for example those of following formula (VII):

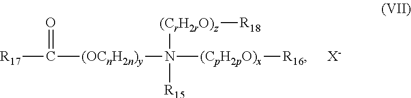

in which:
R₁₅ is chosen from C₁-C₆ alkyl radicals and C₁-C₆ hydroxyalkyl or dihydroxyalkyl radicals;
R₁₆ is chosen from:
   the

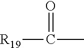

radical
saturated or unsaturated and linear or branched C₁-C₂₂ hydrocarbon radicals R₂₀,
the hydrogen atom,
R₁₈ is chosen from:
   the

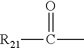

radical
saturated or unsaturated and linear or branched C₁-C₆ hydrocarbon radicals R₂₂,
the hydrogen atom,
R₁₇, R₁₉ and R₂₁, which are identical or different, are chosen from saturated or unsaturated and linear or branched C₇-C₂₁ hydrocarbon radicals;
n, p and r, which are identical or different, are integers with values of from 2 to 6;
y is an integer with a value of from 1 to 10;
x and z, which are identical or different, are integers with values of from 0 to 10;
X⁻ is a simple or complex and organic or inorganic anion;

with the proviso that the sum x+y+z has a value of from 1 to 15, that, when x has a value of 0, then R₁₆ denotes C₂₀ and that, when z has a value of 0, then R₁₈ denotes R₂₂.

The R₁₅ alkyl radicals can be linear or branched and more particularly linear.

R₁₅ preferably denotes a methyl, ethyl, hydroxyethyl or dihydroxypropyl radical and more particularly a methyl or ethyl radical.

The sum x+y+z advantageously has a value of from 1 to 10.

When R₁₆ is a hydrocarbon R₂₀ radical, it can be long and can have from 12 to 22 carbon atoms or can be short and can have from 1 to 3 carbon atoms.

When R₁₈ is a hydrocarbon R₂₂ radical, it preferably has from 1 to 3 carbon atoms.

R₁₇, R₁₉ and R₂₁, which are identical or different, are advantageously chosen from saturated or unsaturated and linear or branched C₁₁-C₂₁ hydrocarbon radicals and more particularly from saturated or unsaturated and linear or branched C₁₁-C₂₁ alkyl and alkenyl radicals.

Preferably, x and z, which are identical or different, have values of 0 or 1.

Advantageously, y is equal to 1.

Preferably, n, p and r, which are identical or different, have values of 2 or 3 and more particularly still are equal to 2.

The anion is preferably a halide (chloride, bromide or iodide) or an alkyl sulphate, more particularly methyl sulphate. However, use may be made of methanesulphonate, phosphate, nitrate, tosylate, an anion derived from an organic acid, such as acetate or lactate, or any other anion compatible with the ammonium comprising an ester functional group.

The X⁻ anion is more particularly still chloride or methyl sulphate.

Use is more particularly made of the ammonium salts of formula (VII) in which:
R₁₅ denotes a methyl or ethyl radical,
x and y are equal to 1;
z is equal to 0 or 1;
n, p and r are equal to 2;
R₁₆ is chosen from:
   the

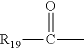

radical
methyl, ethyl or C₁₄-C₂₂ hydrocarbon radicals
the hydrogen atom;
R₁₈ is chosen from:
   the

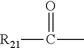

radical
the hydrogen atom;
R₁₇, R₁₉ and R₂₁/which are identical or different, are chosen from saturated or unsaturated and linear or branched C₁₃-C₁₇ hydrocarbon radicals and preferably from saturated or unsaturated and linear or branched C₁₃-C₁₇ alkyl and alkenyl radicals.

The hydrocarbon radicals are advantageously linear.

Preference is given, among quaternary ammonium salts of formula (IV), to, on the one hand, tetraalkylammonium chlorides, such as, for example, dialkyldimethylammonium or alkyltrimethylammonium chlorides, in which the alkyl radical comprises approximately from 12 to 22 carbon atoms, in particular behenyltrimethylammonium, distearyldimethylammonium, cetyltrimethylammonium or benzyldimethylstearylammonium chlorides, or, on the other hand, palmitylamidopropyltrimethylammonium chloride or stearamidopropyldimethyl(myristyl acetate) ammonium chloride, sold under the name “Ceraphyl 70” by Van Dyk.

Mention may be made, for example, of the compounds of formula (V), such as diacyloxyethyldimethylammonium, diacyloxyethyl(hydroxyethyl)methylammonium, monoacyloxyethyl-di(hydroxyethyl)methylammonium, triacyloxyethylmethylammonium or monoacyloxyethyl-(hydroxyethyl)dimethylammonium salts (in particular chloride or methyl sulphate) and their mixtures. The acyl radicals preferably have from 14 to 18 carbon atoms and originate more particularly from a vegetable oil, such as palm oil or sunflower oil. When the compound comprises several acyl radicals, the latter can be identical or different.

These products are obtained, for example, by direct esterification of optionally oxyalkylenated triethanolamine, triisopropanolamine, alkyldiethanol-amine or alkyldiisopropanolamine with fatty acids or with mixtures of fatty acids of vegetable or animal origin or by transesterification of their methyl esters. This esterification is followed by quaternization using an alkylating agent, such as an alkyl halide (preferably methyl or ethyl halide), a dialkyl sulphate (preferably dimethyl or diethyl sulphate), methyl methanesulphonate, methyl para-toluenesulphonate, glycol chlorohydrin or glycerol chlorohydrin.

Such compounds are sold, for example, under the names Dehyquart by Cognis, Stepanquat by Stepan, Noxamium by CECA and Rewoquat WE 18 and Rewoquat W75 by Degussa.

Use may also be made of the ammonium salts comprising at least one ester functional group described in U.S. Pat. No. 4,874,554 and U.S. Pat. No. 4,137,180.

Di(quaternary ammonium) salts of formula (VI) suitable for the invention comprise in particular propanetallowediammonium dichloride.

The amount (as active material) of additional foaming surfactant(s) in the composition according to the invention can range, for example, from 0.1 to 10% by weight, preferably from 0.2 to 7% by weight and better still from 0.5 to 5% by weight, with respect to the total weight of the composition.

The composition can comprise, apart from the anionic and amphoteric surfactants and the additional surfactants, a polymeric quaternary ammonium salt (distinct from the preceding surfactants) and/or a cationic galactomannan gum, preferably a cationic guar gum.

These compounds make it possible to increase the amount of foam and to obtain a feeling of softness and of comfort on the skin (maintenance of the moisturizing).

Polymeric quaternary ammonium salts are cationic or amphoteric polymers comprising at least one quaternized nitrogen atom. Mention may in particular be made, as polymeric quaternary ammonium salts, of the Polyquaternium products (CTFA name), which contribute softness and smoothness to the foaming cream. These polymers can preferably be chosen from the following polymers:
Polyquaternium-5, such as the product Merquat 5 sold by Calgon;
Polyquaternium-6, such as the product Salcare SC 30 sold by Ciba and the product Merquat 100 sold by Calgon;
Polyquaternium-7, such as the products Merquat S, Merquat 2200 and Merquat 550 sold by Calgon and the product Salcare SC 10 sold by Ciba;
Polyquaternium-10, such as the product Polymer JR400 sold by Amerchol;
Polyquaternium-11, such as the products Gafquat 755, Gafquat 755N and Gafquat 734 sold by ISP;
Polyquaternium-15, such as the product Rohagit KF 720 F sold by Röhm;
Polyquaternium-16, such as the products Luviquat FC905, Luviquat FC370, Luviquat HM552 and Luviquat FC550 sold by BASF;
Polyquaternium-22, such as the product Merquat 280 sold by Calgon;
Polyquaternium-28, such as the product Styleze CC10 sold by ISP;
Polyquaternium-39, such as the product Merquat Plus 3330 sold by Calgon;
Polyquaternium-44, such as the product Luviquat Care sold by BASF;
Polyquaternium-46, such as the product Luviquat Hold sold by BASF;
Polyquaternium-47, such as the product Merquat 2001 sold by Calgon.

Preferably, the quaternary ammonium salts are chosen from Polyquaternium-7, Polyquaternium-10, Polyquaternium-39, Polyquaternium-47 and their mixtures.

The cationic galactomannan gums preferably have a cationic charge density of less than or equal to 1.5 meq/g and more particularly of between 0.1 and 1 meq/g.

Generally, within the meaning of the present invention, the term “cationic galactomannan gum” is understood to mean any galactomannan gum comprising cationic groups and/or groups which can be ionized to give cationic groups.

The preferred cationic groups are chosen from those comprising primary, secondary, tertiary and/or quaternary amine groups.

The cationic galactomannan gums used generally have a weight-average molecular weight of between 500 and 5×10⁶ approximately and preferably of between 10³ and 3×10⁶ approximately.

The cationic galactomannan gums which can be used according to the present invention are, for example, gums comprising tri(C₁-C₄)alkylammonium cationic groups. Preferably, from 2 to 3% by number of the hydroxyl functional groups of these gums carry trialkylammonium cationic groups.

Mention may very particularly be made, among these trialkylammonium groups, of trimethylammonium and triethylammonium groups.

More preferably still, these groups represent from 5 to 20% by weight of the total weight of the modified galactomannan gum.

According to the invention, use is preferably made of a guar gum comprising hydroxypropyltrimethylammonium groups, that is to say a guar gum modified, for example, with 2,3-epoxypropyltrimethylammonium chloride.

These galactomannan gums, in particular guar gums, modified with cationic groups are products already known in themselves and are, for example, described in U.S. Pat. No. 3,589,578 and U.S. Pat. No. 4,031,307. Furthermore, such products are sold in particular under the trade names of Jaguar C13S, Jaguar C15, Jaguar C17 and Jaguar C162 by Meyhall.

The polymeric quaternary ammonium salts and/or the cationic galactomannan gums can be in an amount (as active material) ranging, for example, from 0.01 to 5% by weight and better still from 0.05 to 1% by weight, with respect to the total weight of the composition.

According to one embodiment, the composition according to the invention comprises an aqueous phase comprising water and optionally one or more water-soluble organic solvents. In this aqueous medium, the amount of water is preferably at least 20% by weight; it preferably ranges from 20 to 95% by weight, better still from 30 to 90% by weight and even better still from 40 to 85% by weight, with respect to the total weight of the composition.

The water-soluble solvents can be chosen from water-soluble lower alcohol(s). The term “lower alcohol” is understood to mean an alcohol comprising from 1 to 8 carbon atoms. Mention may be made, as lower alcohols, for example, of ethanol, isopropanol, butanol and their mixtures. When they are present in the composition of the invention, the water-soluble lower alcohol(s) can be in an amount ranging from 0.01 to 40% by weight.

The aqueous phase can also comprise polyols (or polyhydric alcohols), such as, for example, glycerol; glycols, such as propylene glycol, butylene glycol, isoprene glycol and polyethylene glycols, such as PEG-8; sorbitol; sugars, such as glucose, fructose, maltose, lactose or sucrose; and their mixtures. The amount of polyols generally ranges from 0.1 to 60% by weight and better still from 0.5 to 50% by weight, with respect to the total weight of the aqueous phase.

According to one embodiment, the composition according to the invention comprises less than 5% by weight of water, preferably less than 3% and better still less than 2%; they can be entirely devoid of water (anhydrous composition). However, it can comprise water-soluble solvents, such as the lower alcohols or the polyols mentioned above.

### Oils

The composition according to the invention can comprise an oil.

Mention may be made, as oils which can be used in the composition of the invention, for example, of:

essential oils,

hydrocarbon oils of animal origin, such as perhydrosqualene;

hydrocarbon oils of vegetable origin, such as liquid triglycerides of fatty acids comprising from 4 to 30 carbon atoms, such as triglycerides of heptanoic acid or octanoic acid, or, for example, sunflower, jojoba, maize, soybean, cucumber, grape seed, sesame, hazelnut, apricot, macadamia, arara, coriander, castor or avocado oils, triglycerides of caprylic/capric acids, such as those sold by Stearineries Dubois or those sold under the names Miglyol 810, 812 and 818 by Dynamit Nobel, or shea butter oil;

synthetic esters and ethers, in particular of fatty acids, such as oils of formulae R¹COOR² and R¹OR² in which R¹ represents the residue of a fatty acid or of a fatty alcohol comprising from 8 to 29 carbon atoms and R² represents a branched or unbranched hydrocarbon chain comprising from 3 to 30 carbon atoms, such as, for example, purcellin oil, 2-octyldodecyl stearate, 2-octyldodecyl erucate, or isostearyl isostearate; hydroxylated esters, such as isostearyl lactate, octyl hydroxystearate, octyldodecyl hydroxystearate, diisostearyl malate, triisocetyl citrate or heptanoates, octanoates or decanoates of fatty alcohols; polyol esters, such as propylene glycol dioctanoate, neopentyl glycol diheptanoate and diethylene glycol diisononanoate; and pentaerythritol esters, such as pentaerythrityl tetraisostearate;

linear or branched hydrocarbons of mineral or synthetic origin, such as volatile or nonvolatile liquid paraffins and their derivatives, liquid petrolatum, polydecenes, isohexadecane, isododecane or hydrogenated polyisobutene, such as Parleam® oil;

silicone oils, such as volatile or nonvolatile polymethylsiloxanes (PDMSs) comprising a linear or cyclic silicone chain which are liquid or pasty at ambient temperature, in particular volatile silicone oils, especially cyclopolydimethylsiloxanes (cyclomethicones), such as cyclohexadimethylsiloxane and cyclopentadimethylsiloxane; polydimethylsiloxanes comprising pendent alkyl, alkoxy or phenyl groups or alkyl, alkoxy or phenyl groups at the end of the silicone chain, which groups have from 2 to 24 carbon atoms; or phenylated silicones, such as phenyl trimethicones, phenyl dimethicones, phenyl(trimethyl-siloxy)diphenylsiloxanes, diphenyl dimethicones, diphenyl(methyldiphenyl)trisiloxanes, (2-phenylethyl)-trimethylsiloxysilicates and polymethylphenylsiloxanes;

their mixtures.

The oil is can be present in a content ranging from 0.5 to 15% by weight, with respect to the total weight of the composition, preferably less than or equal to 1 to 12% by weight.

The compositions of the invention can also comprise adjuvants generally used in the cosmetics field. Mention may be made, as adjuvants, for example, of fragrances, preservatives, sequestering agents (EDTA), pigments, fillers, in particular exfoliating fillers, soluble dyes, sunscreens, cosmetic or dermatological active principles, such as moisturizing agents, for example hyaluronic acid; ceramides; water-soluble or fat-soluble vitamins, such as vitamin C and its derivatives, such as vitamin CG; antiseptics; antiseborrhoeics; antimicrobials, such as benzoyl peroxide, salicylic acid, triclosan or azeleic acid; or optical brighteners. The amounts of these various adjuvants are those conventionally used in the field under consideration, for example from 0.01 to 5% of the total weight of the composition. These adjuvants and their concentrations have to be such that they do not modify the property desired for the composition of the invention.

Mention may be made, as exfoliants, for example, of exfoliating or scrubbing particles of mineral, vegetable or organic origin. Thus, use may be made, for example, of polyethylene beads or powder, nylon powder, polyvinyl chloride powder, pumice, ground materials derived from apricot kernels or walnut shells, sawdust, glass beads, alumina and their mixtures. These particles can be present in an amount ranging, for example, from 0.5 to 40% by weight, preferably from 1 to 20% by weight and better still from 1 to 10% by weight, with respect to the total weight of the composition. When the composition comprises exfoliating particles, it can constitute in particular a composition for scrubbing the skin of the face or body.

The compositions of the invention can also comprise polymers, in particular anionic and nonionic polymers.

Mention may in particular be made, as anionic polymers, of those comprising at least one hydrophobic chain and in particular those derived from acrylic or methacrylic acid, such as the acrylates/steareth-20 methacrylate copolymer sold under the name Aculyn 22 by Röhm & Haas (CTFA name: acrylates/steareth-30 methacrylate copolymer); the oxyethylenated (25 EO) (meth)acrylic acid/ethyl acrylate/behenyl methacrylate terpolymer, as an aqueous emulsion, sold under the name Aculyn 28 by Röhm & Haas; the oxyethylenated (20 EO) acrylic acid/monocetyl itaconate copolymer, as a 30% aqueous dispersion, sold under the name Structure 3001 by National Starch; the oxyethylenated (20 EO) acrylic acid/monostearyl itaconic copolymer, as a 30% aqueous suspension, sold under the name Structure 2001 by National Starch; the acrylates/acrylate copolymer modified with polyoxyethylenated (25 EO) C₁₂-C₂₄ alcohols, in the form of a latex containing 30-32% of copolymer, sold under the name Synthalen W2000 by 3V SA, and the copolymers sold under the Pemulen or Carbopol names by Noveon, such as the acrylate/C₁₀-C₃₀-alkylacrylate copolymer, for example the products Pemulen TR1 or Carbopol 1382 (CTFA name: acrylates/C10-30 alkyl acrylate crosspolymer).

The composition can also comprise polymers comprising at least one monomer comprising a sulpho group and in particular polymers and copolymers of 2-acrylamido-2-methylpropanesulphonic acid (AMPS), such as:

the crosslinked and neutralized homopolymer of 2-acrylamido-2-methylpropanesulphonic acid, sold by Clariant under the trade name “Hostacerin AMPS” (CTFA name: ammonium polyacrylodimethyltauramide);

crosslinked anionic copolymers of acrylamide or methacrylamide and of 2-acrylamido-2-methylpropanesulphonic acid, in particular those provided in the form of a W/O emulsion, such as those sold under the name of Sepigel 305 by Seppic (CTFA name: polyacrylamide/C13-14 isoparaffin/laureth-7) or under the name of Simulgel 600 by Seppic (CTFA name: acrylamide/sodium acryloyldimethyltaurate copolymer/isohexadecane/polysorbate 80),

copolymers of (meth)acrylic acid or (meth)acrylate and of 2-acrylamido-2-methylpropanesulphonic acid, in particular those provided in the form of a W/O emulsion, such as those sold under the name Simulgel NS by Seppic (sodium 2-acrylamido-2-methylpropane-sulphonate/hydroxyethyl acrylate copolymer as a 40% inverse emulsion in polysorbate 60 and squalene) (CTFA name: hydroxyethyl acrylate/sodium acryloyldimethyltaurate copolymer/squalene/polysorbate 60) or those sold under the name of Simulgel EG by Seppic (acrylic acid/2-acrylamido-2-methylpropanesulphonic acid in the form of a sodium salt copolymer, as a 45% inverse emulsion in isohexadecane/water) (CTFA name: sodium acrylate/sodium acryloyldimethyltaurate copolymer/isohexadecane/polysorbate 80),

copolymers of 2-acrylamido-2-methylpropanesulphonic acid and of vinylpyrrolidone or vinylformamide, such as the products sold under the Aristoflex AVC names by Clariant,

hydrophobic modified AMPS polymers, such as in particular the copolymer of AMPS and of ethoxylated C₁₂-C₁₄ alkyl methacrylate (noncrosslinked copolymer obtained from Genapol LA-070 and AMPS) (CTFA name: ammonium acryloyldimethyltaurate/laureth-7 methacrylate copolymer), sold under the name Aristoflex LNC by Clariant, and the copolymer of AMPS and of ethoxylated (25 EO) stearyl methacrylate (copolymer crosslinked with trimethylolpropane triacrylate, obtained from Genapol T-250 and AMPS) (CTFA name: ammonium acryloyldimethyltaurate/steareth-25 methacrylate crosspolymer), sold under the name Aristoflex HMS by Clariant.

These polymers can be in an amount (as active material) ranging, for example, from 0.05 to 10% by weight and better still from 0.1 to 5% by weight, with respect to the total weight of the composition.

The compositions according to the invention can be used on all keratinous substances, such as the skin, scalp, hair, eyelashes, eyebrows, nails or mucous membranes, in particular as hygiene product, for example as product for cleansing the skin, mucous membranes and/or hair, in particular as product for cleansing and/or removing makeup from the skin (or the face and/or the body), as shower product (two-in-one product), as shampoo and conditioner, as shaving product, as rinse-off mask and as exfoliating product (also known as desquamating or deep cleansing agent), both for the face and for the body or for the hands, after addition of exfoliating particles.

A further subject-matter of the invention is the cosmetic use of the composition as defined above as product for cleansing and/or removing makeup from the skin, as shower product, as shampoo, as conditioner, as shaving product, as rinse-off mask or as exfoliating product.

Another subject-matter of the invention is a method for cleansing a keratinous substance, such as the skin, scalp, hair, eyelashes, eyebrows, nails or mucous membranes, wherein a composition as defined above is applied to the keratinous substance and in that it is rinsed off.

The keratinous substance is preferably the skin.

The compositions according to the invention can, for example, be used in the following way:

1) when the compositions according to the invention constitute mild cleansing products for the face, they can be used in the following way:

the product is brought to a foam in the hands with water

the foam is applied to the face

the face is cleansed

rinsing is carried out with water

2) when the compositions according to the invention constitute makeup-removing products, they can be used as indicated above but they can also be applied dry to the face, then massaged, until satisfactory removal of makeup is obtained, and rinsed off with water, or else they can be applied using a piece of cotton wool.
3) they can be used as two-in-one shower product, as shampoo and/or conditioner, as rinse-off mask or as shaving product, in the usual way in which these products are used.

The examples which follow serve to illustrate the invention without, however, exhibiting a limiting nature. The amounts shown are as % by weight, unless otherwise mentioned, and they correspond, unless otherwise mentioned, to the amount of starting material and not to the amount of active material. The names of the compounds used are shown as CTFA name, as chemical name or as commercial name.

### EXAMPLES

In the examples which follow, the foaming performance (foaming qualities) are evaluated according to the following protocol:

Before any use of the product, the hands are washed with household soap and then suitably rinsed and dried. The protocol followed is then as follows:

1—the hands are wetted by passing them under running water and shaking them three times to remove the excess water from them,

2—1 g of product is placed in the hollow of one of the hands,

3—the product is worked between the two palms for 10 seconds,

4—2 ml of water are added and the product is again worked for 10 seconds,

5—the hands are rinsed under water,

6—they are dried.

The following criteria are evaluated:
volume of foam: the grade assigned increases as the volume increases,
size of the bubbles composing the foam: the grade assigned increases as the bubbles become larger,
density: consistency, behaviour of the foam: the grade assigned increases as the density increases.

They are graded on a scale from 0 to 10. For a given criterion, a difference in grade between two products is regarded as existing when the difference between two grades is greater than 0.5.

The evaluation panel is composed of 3 trained experts. The mean of the 3 grades makes it possible to compare the compositions according to each of the criteria.

### Examples 1 to 3

The following compositions are prepared:

| | | Example | | Example | | Example |
|---|---|---|---|---|---|---|
| | Example 1 | 1′ | Example 2 | 2′ | Example 3 | 3′ |
| | (invention) | (comparative) | (invention) | (comparative) | (invention) | (comparative) |
| Sodium lauryl ether | 18.6% | 18.6% | — | — | — | — |
| sulphate (70% AM) (1) | | | | | | |
| Laureth-5 carboxylic acid | — | — | 14.44% | 14.44% | — | — |
| (90% AM) (2) | | | | | | |
| Coco betaine (30% AM) (3) | — | — | — | — | 43.33% | 43.33% |
| Sodium salt of 3-hydroxy- | 6.67% | — | 6.67% | — | 6.67% | — |
| 2 pentylcyclopentaneacetic | | | | | | |
| acid at 30% in a water/ | | | | | | |
| dipropylene glycol (70/30) | | | | | | |
| mixture | | | | | | |
| Water | 74.73% | 81.4% | 69.75% | 75.56% | 50% | 56.67% |
| 10% Sodium hydroxide in | — | — | 9.14% | 10% | — | — |
| water | | | | | | |
| Appearance | Clear | Clear | Clear | Clear | Clear | Clear |
| | transparent | transparent | transparent | transparent | transparent | transparent |
| | solution | solution | solution | solution | solution | solution |
| pH | 7.2 | 8 | 6.7 | 6.8 | 6.9 | 7.5 |
| Grades: | | | | | | |
| Before addition of water: | | | | | | |
| Volume of foam | 3.2 | 2.7 | 3.5 | 2.2 | 4.5 | 3.5 |
| Size of bubbles | 3.8 | 3.7 | 3.8 | 2.3 | 6.5 | 6.3 |
| Density of the foam | 7.5 | 6.2 | 7 | 5.3 | 7.3 | 6.8 |
| After addition of water: | | | | | | |
| Volume of foam | 6.7 | 5.7 | 6.3 | 4.7 | 7.7 | 6.5 |
| Size of bubbles | 5 | 5.5 | 4 | 4.2 | 6.7 | 6.3 |
| Density of the foam | 6.8 | 5.3 | 6.7 | 5.5 | 9.7 | 9 |

(1) Texapon AOS 225 UP (Cognis)
(2) Akypo RLM 45 CA (Kao)
(3) Genagen KB (Clariant)

Procedure: The mixtures of surfactants are produced at ambient temperature using a magnetic bar. The 3-hydroxy-2-pentylcyclopentaneacetic acid salt, if present, is added under cold conditions to the solution of surfactants.

These examples show that the addition of jasmonic acid derivative increases the volume and the density of the foam, at an identical bubble size, in anionic or amphoteric surface-active bases.

### Examples 4 and 4′

### Foaming Cleansing Creams

| | | | Example 4′ |
|---|---|---|---|
| | | Example 4 | (compar- |
| | Phase | (invention) | ative) |
| Water | A1 | 8.42% | 8.42% |
| Carbomer (1) | A2 | 0.25% | 0.25% |
| Glycerol | A3 | 39.3% | 39.3% |
| Guar hydroxypropyl trimonium chloride | A4 | 0.25% | 0.25% |
| (30% AM) (2) | | | |
| Sodium cocoyl glycinate (52% AM) (3) | B1 | 43.33% | 43.33% |
| Glycol distearate | B2 | 1.8% | 1.8% |
| Stearic acid | B3 | 1.1% | 1.1% |
| Polyquaternium-39 (10% AM) (4) | C | 2.5% | 2.5% |
| Citric acid (20%) | D | 2.55% | 2.55% |
| Fragrance | E | 0.5% | 0.5% |
| Sodium salt of 3-hydroxy-2-pentyl- | F | 6.67% | — |
| cyclopentaneacetic acid at 30% in a | | | |
| water/dipropylene glycol (70/30) | | | |
| mixture | | | |

| Appearance | White | White |
|---|---|---|
| | cream | cream |
| pH | 7.0 | 7.0 |
| Sensory grades | | |
| Before addition of water: | | |
| Volume of foam | 5.3 | 6.5 |
| Size of bubbles | 2.3 | 2.5 |
| Density of the foam | 7 | 8 |
| After addition of water: | | |
| Volume of foam | 7.5 | 8.5 |
| Size of bubbles | 2.5 | 2.5 |
| Density of the foam | 7.5 | 8 |

(1) Carbopol Ultrez 10 (Lubrizol)
(2) Jaguar C 162 (Rhodia)
(3) Amilite GCS-12K (Ajinomoto)
(4) Merquat Plus 3330 (Nalco)

### Procedure:

1—The phase A=A1+A2+A3+A4 is prepared under a deflocculator at ambient temperature.
2—The phase B=B1+B2+B3 is heated to 70-75° C. on a water bath.
3—The aqueous phase A is gradually introduced into the hot fatty phase B and the mixture is homogenized at 50-55° C. for 10 min.
4—C, D, E and F are incorporated.
5—The mixture is cooled under the deflocculator.

These examples show that the presence of the salt of 3-hydroxy-2-pentylcyclopentaneacetic acid significantly improves the foaming quality of a composition comprising a surfactant composition comprising an anionic surfactant.

The composition of Example 4 according to the invention gives a copious and dense foam, is rinsed off well and is well tolerated by the skin. It can advantageously be used for cleansing the skin of the face.

### Examples 5 and 5′

### Foaming Liquid Soap Creams

| | Example 5 | Example 5′ |
|---|---|---|
| Sodium lauryl ether sulphate (70% AM) (1) | 30.33% | 30.33% |
| Alkyl polyglucoside (52% AM) (2) | 7.5% | 7.5% |
| Water | 62.17% | 55.5% |
| Sodium salt of 3-hydroxy-2-pentylcyclo- | | 6.67% |
| pentaneacetic acid at 30% in a water/ | | |
| dipropylene glycol (70/30) mixture | | |
| Citric acid (10% AM) | q.s. for pH | q.s. for pH |
| Appearance | Transparent | Transparent |
| | solution | solution |
| pH | 7.1 | 7.2 |
| Sensory grades | | |
| Before addition of water: | | |
| Volume of foam | 4.5 | 7 |
| Size of bubbles | 5.3 | 4.3 |
| Density of the foam | 5.5 | 7.3 |
| After addition of water: | | |
| Volume of foam | 6 | 9.5 |
| Size of bubbles | 6.5 | 5 |
| Density of the foam | 2.5 | 7 |

(1) Texapon AOS 225 UP (Cognis)
(2) Plantacare 818 UP (Cognis)

The presence of the salt of 3-hydroxy-2-pentylcyclopentaneacetic acid significantly improves the foaming quality of the composition according to the invention.

The above written description of the invention provides a manner and process of making and using it such that any person skilled in this art is enabled to make and use the same, this enablement being provided in particular for the subject matter of the appended claims, which make up a part of the original description.

As used herein, the words “a” and “an” and the like carry the meaning of “one or more.”

The phrases “selected from the group consisting of,” “chosen from,” and the like include mixtures of the specified materials. Terms such as “contain(s)” and the like are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition comprising:
(1) at least one surfactant selected from foaming anionic surfactants and foaming amphoteric surfactants, and
(2) at least one compound of formula (I): 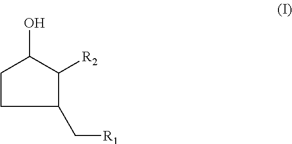
in which:
R₁ represents a COOR₃ radical, R₃ denoting a hydrogen atom or a C₁-C₄ alkyl radical optionally substituted by one or more hydroxyl groups;
R₂ represents a hydrocarbon radical which is saturated or unsaturated, which is linear and which has from 1 to carbon atoms or which is branched or cyclic and which has from 3 to 18 carbon atoms;
and their optical isomers, and corresponding salts.

2. The composition according to claim 1, wherein R₁ denotes a radical chosen from —COOH, —COOMe, —COO—CH₂—CH₃, —COO—CH₂—CH(OH)—CH₂OH, —COOCH₂—CH₂—CH₂OH and —COOCH₂—CH(OH)—CH₃.

3. The composition according to claim 1, wherein R₁ denotes a —COOH radical.

4. The composition according to claim 1, wherein R₂ denotes a saturated or unsaturated linear hydrocarbon radical having from 2 to 7 carbon atoms.

5. The composition according to claim 1, wherein R₂ is a pentyl, pentenyl, hexyl or heptyl radical.

6. The composition according to claim 1, wherein the compound of formula (I) is chosen from 3-hydroxy-2-[(2Z)-2-pentenyl]cyclopentaneacetic acid and 3-hydroxy-2-pentylcyclopentaneacetic acid.

7. The composition according to claim 1, wherein the salts of the compounds of formula (I) are chosen from alkali metal salts, alkaline earth metal salts, metal salts, salts of ammonium of formula NH₄⁺, quaternary ammonium salts, organic amine salts, lysine salts and arginine salts.

8. The composition according to claim 1, wherein the compound of formula (I) present is from 0.1 to 15% by weight with respect to the total weight of the composition.

9. The composition according to claim 1, wherein the at least one surfactant is present in an amount of 0.1 to 40% by weight with respect to the total weight of the composition.

10. The composition according to claim 1, comprising an anionic surfactant chosen from anionic derivatives of proteins of vegetable origin or of silk proteins, phosphates and alkyl phosphates, carboxylates, sulphosuccinates, amino acid derivatives, alkyl sulphates, alkyl ether sulphates, sulphonates, isethionates, taurates, alkyl sulphoacetates, polypeptides, anionic derivatives of alkyl polyglucosides, soaps (salts of fatty acids), and mixtures thereof.

11. The composition according to claim 1, comprising an anionic surfactant chosen from alkyl sulphates, alkyl ether sulphates, isethionates, amino acid derivatives and mixtures thereof.

12. The composition according to claim 11, comprising a surfactant chosen from betaines, N-alkyl-amidobetaines and their derivatives, sultaines, alkyl polyaminocarboxylates, alkylamphoacetates, their mixtures thereof.

13. The composition according to claim 1, comprising a surfactant chosen from betaines, alkylamphoacetates, and mixtures thereof.

14. The composition according to claim 1, further comprising at least one polymeric quaternary ammonium salt different from said at least one surfactant selected from foaming anionic surfactants and foaming amphoteric surfactants and/or at least one cationic galactomannan gum.

15. A method for cleansing a keratinous substance, comprising applying a composition according to claim 1 to the keratinous substance and rinsing the composition off the keratinous substance.
